(19) United States
(12) Patent Application Publication
(10) Pub. No.: US 2023/0200924 A1
(43) Pub. Date: Jun. 29, 2023

(54) DISPOSABLE GLOVE DISPENSER

(71) Applicant: Robert Guzman, Lakeland, FL (US)

(72) Inventor: Robert Guzman, Lakeland, FL (US)

(21) Appl. No.: 17/543,428

(22) Filed: Dec. 6, 2021

(51) Int. Cl. A61B 42/50; A61B 42/40

(52) U.S. Cl. CPC A61B 42/50; A61B 42/40

(57) ABSTRACT

Methods, systems and computer readable media to dispense disposable gloves are described.

### TECHNICAL FIELD

Embodiments relate generally to disposable item dispensers, and more particularly, to methods, systems and computer readable media for disposable glove dispensers.

### BACKGROUND

Disposable gloves are used in a number of settings including health care, food preparation and service, law enforcement, education, childcare, elder care, and many others. Disposable glove users may need to put on multiple pairs of gloves per day and may have a need to receive disposable gloves and put them without contacting the glove dispenser (e.g., once hands have been washed and dried, etc.).

Some implementations were conceived in light of the above-mentioned needs, problems and/or limitations, among other things.

### SUMMARY

Some implementations can include a glove dispenser comprising a housing containing a disposable glove supply configured to store one or more disposable gloves, a glove dispensing bay configured to retrieve one or more disposable gloves from the disposable glove supply, a glove opener and inflator configured to at least partially open and partially inflate the one or more disposable gloves received in the glove dispensing bay, and a control unit configured to control the glove dispenser.

In some implementations, the control unit is configured to perform operations including detecting when a user inserts one or both hands into the glove dispensing bay, prior to the user inserting one or both hands into the glove dispensing bay, causing a glove opener and inflator to retrieve one or more gloves from the disposable glove supply, and as the one or more hands of the user enters the glove dispensing bay, causing the glove opener and inflator to mechanically open the one or more gloves at least partially and causing the inflator to blow air into the glove to partially inflate the one or more gloves such that the user is aided in inserting the one or more hands into the one or more gloves.

In some implementations, the air used to at least partially inflate the one or more gloves is supplied by an air source comprising one of an air pump or a compressed air source. In some implementations, the disposable glove dispenser further comprises an ultraviolet (UV) or other proximity sensor to detect proximity of the one or more hands of a user. In some implementations, the glove dispenser is configured to dispense a single size of disposable glove.

In some implementations, the glove dispenser is configured to dispense multiple sizes of disposable gloves.

Some implementations can include a method to operate and control a disposable glove dispenser. The method can include detecting, at the disposable glove dispenser, a user's hand approaching or entering a glove dispensing bay of the disposable glove dispenser, loading one or more disposable gloves into the glove dispensing bay by mechanically retrieving the one or more gloves from a disposable glove supply, and partially opening the one or more disposable gloves are partially opened. The method can also include activating an inflation air source to partially inflate the one or more gloves, detecting a withdrawal of the one or more hands of the user from the glove dispensing bay, and deactivating the inflation air source.

The method can further include resetting the disposable glove dispenser for a next dispensing operation. The method can also include receiving, at the disposable glove dispensing system a glove size selection.

In some implementations, the glove size selection is received from one of a control panel, a voice command, or by inserting the one or more hands into a slot associated with a particular size of disposable glove. In some implementations, the disposable glove dispenser includes a UV or other proximity sensor to detect the proximity of the one or more hands of the user.

In some implementations, the disposable glove supply is within the disposable glove dispenser. In some implementations, the disposable glove supply is external to the disposable glove dispenser. In some implementations, the partial opening is performed mechanically.

### BRIEF DESCRIPTION OF THE DRAWINGS

FIG. 1 is a diagram showing an example disposable glove dispenser in accordance with some implementations.

FIG. 2 is a diagram showing an example disposable glove dispenser in accordance with some implementations.

FIG. 3 is a diagram showing a hand inside a partially inflated disposable glove in accordance with some implementations.

FIG. 4 is a diagram showing a disposable glove on a hand in accordance with some implementations.

FIG. 5 is a flowchart showing an example method to operate and control a disposable glove dispenser in accordance with some implementations.

FIG. 6 is a diagram of an example computing device configured for electronic employment document control in accordance with at least one implementation.

FIG. 7 is a diagram showing the front of an example disposable glove dispenser in accordance with some implementations.

FIG. 8 is a diagram showing the front of an example glove dispenser in accordance with some implementations.

### DETAILED DESCRIPTION

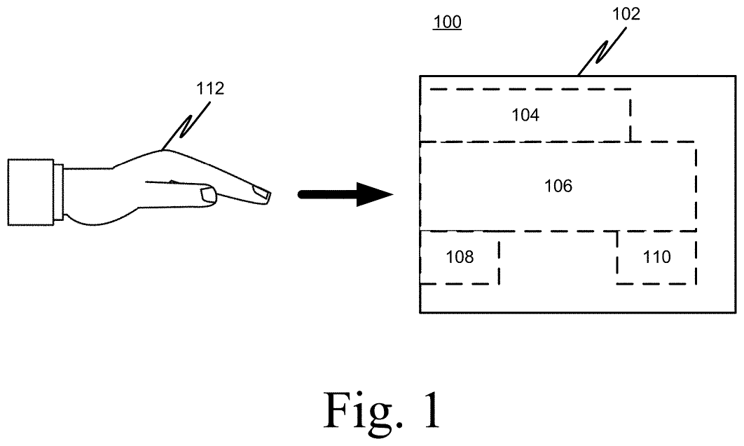

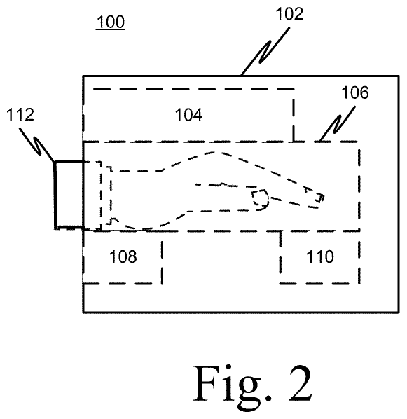

FIGS. 1 and 2 are diagrams showing an example disposable glove dispenser 100 in accordance with some implementations. The dispenser 100 includes a housing 102 containing a disposable glove supply 104, a glove dispensing bay 106, a glove opener and inflator 108, and a control unit 110.

In operation, a user inserts one or both hands 112 into the glove dispensing bay 106 (as shown in FIG. 2) , where, prior to the user's hand being inserted, the glove opener and inflator has retrieved a glove from the disposable glove supply 104 and as the user's hand 112 enters the glove dispensing bay 106, the glove opener and inflator 108 mechanically opens the glove at least partially and the inflator blows air (from an air source such as a small air pump or compressed air container) into the glove to partially inflate the glove such that the user is aided in inserting his or her hand 112 into the glove.

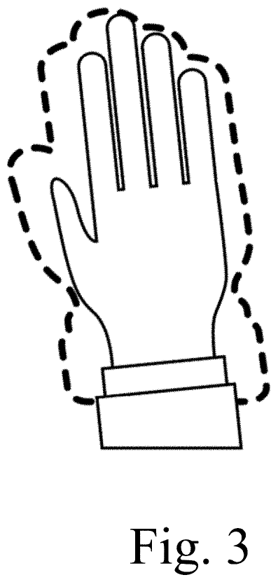

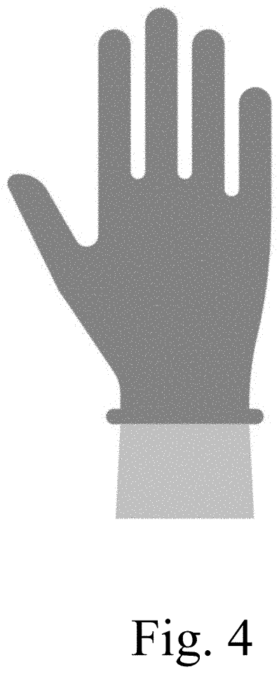

FIG. 3 is a diagram showing a hand inside a partially inflated disposable glove in accordance with some implementations. The partial inflation permits the users hand to more easily be inserted into the disposable glove. FIG. 4 is a diagram showing a disposable glove on a hand in accordance with some implementations.

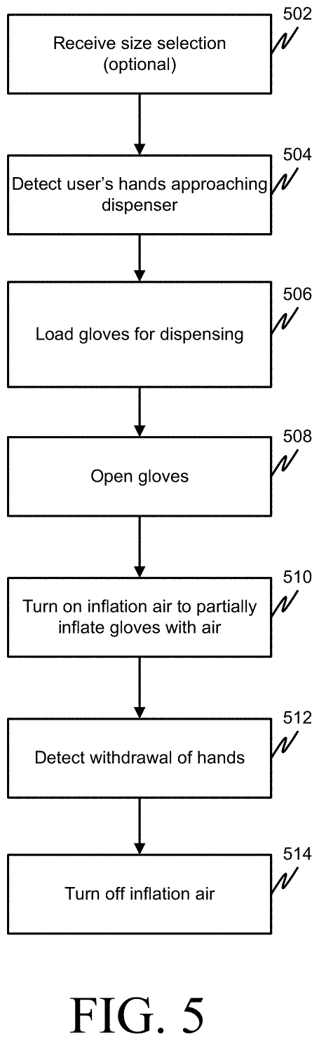

FIG. 5 is a flowchart of an example method to operate and control a disposable glove dispenser (e.g., 100). Processing begins at 502, where the disposable glove dispensing system receives an optional size selection. In some implementations, the dispenser may only dispense a single size glove and in other implementations, the dispenser my dispense multiple sizes of gloves (e.g., small, medium, and large). The size selection can be in the form of a selection from a control panel, a voice command, or by inserting the hands into a slot associated with a particular size glove. Processing continues to 1504.

At 504, the dispenser detects the user's hands approaching or entering the glove dispensing bay of the dispenser. For example, the dispenser can include a UV or other proximity sensor to detect the proximity of the user's hands. Processing continues to 506.

At 506, gloves are loaded for dispensing by mechanically retrieving gloves from the glove supply within the dispenser. Processing continues to 508.

At 508, the gloves are partially opened (e.g., mechanically opened). Processing continues to 510.

At 510, the inflation air source is turned on to partially inflate the gloves. Processing continues to 512.

At 512, the dispenser detects the withdrawal of the user's hands from the dispensing bay. Processing continues to 514.

At 514, the inflation air source is turned off.

The dispenser then resets for the next dispensing operation.

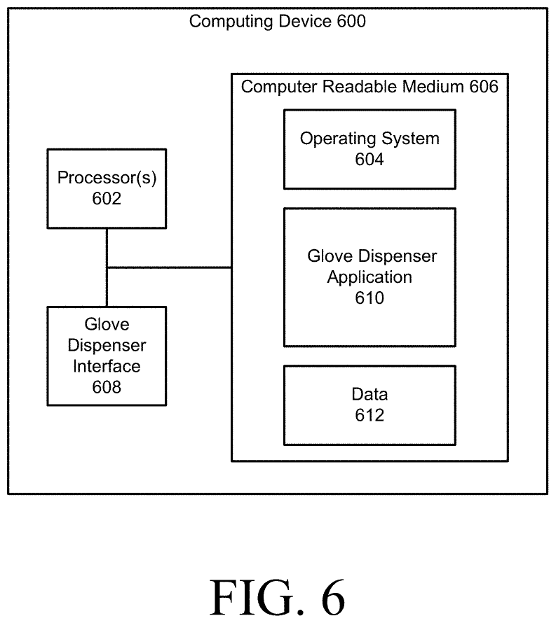

FIG. 6 is a diagram of an example computing device 600 in accordance with at least one implementation. The computing device 600 includes one or more processors 602, nontransitory computer readable medium 606 and network interface 608. The computer readable medium 606 can include an operating system 604, an application 610 for disposable glove dispenser control and a data section 612 (e.g., for storing inventory and dispensing data, etc.).

In operation, the processor 602 may execute the application 610 stored in the computer readable medium 606. The application 610 can include software instructions that, when executed by the processor, cause the processor to perform operations to control a disposable glove dispenser in accordance with the present disclosure (e.g., performing associated functions described above and in FIG. 5).

The application program 610 can operate in conjunction with the data section 612 and the operating system 604.

It will be appreciated that the modules, processes, systems, and sections described above can be implemented in hardware, hardware programmed by software, software instructions stored on a nontransitory computer readable medium or a combination of the above. A system as described above, for example, can include a processor configured to execute a sequence of programmed instructions stored on a nontransitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C, C++, C#.net, assembly or the like. The instructions can also comprise code and data objects provided in accordance with, for example, the Visual Basic™ language, or another structured or object-oriented programming language. The sequence of programmed instructions, or programmable logic device configuration software, and data associated therewith can be stored in a nontransitory computer-readable medium such as a computer memory or storage device which may be any suitable memory apparatus, such as, but not limited to ROM, PROM, EEPROM, RAM, flash memory, disk drive and the like.

Furthermore, the modules, processes systems, and sections can be implemented as a single processor or as a distributed processor. Further, it should be appreciated that the steps mentioned above may be performed on a single or distributed processor (single and/or multi-core, or cloud computing system). Also, the processes, system components, modules, and sub-modules described in the various figures of and for embodiments above may be distributed across multiple computers or systems or may be co-located in a single processor or system. Example structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and/or a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a PLD, PLA, FPGA, PAL, or the like. In general, any processor capable of implementing the functions or steps described herein can be used to implement embodiments of the method, system, or a computer program product (software program stored on a nontransitory computer readable medium).

Furthermore, embodiments of the disclosed method, system, and computer program product (or software instructions stored on a nontransitory computer readable medium) may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a VLSI design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of the software engineering and computer networking arts.

Moreover, embodiments of the disclosed method, system, and computer readable media (or computer program product) can be implemented in software executed on a programmed general-purpose computer, a special purpose computer, a microprocessor, a network server or switch, or the like.

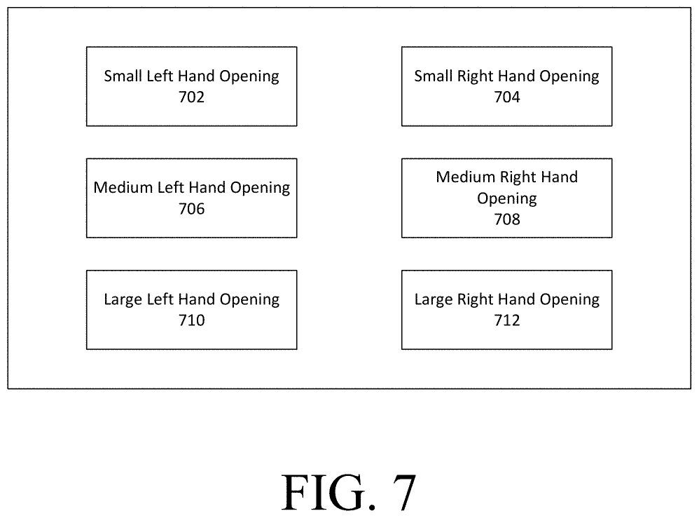

FIG. 7 is a diagram showing the front of an example disposable glove dispenser having two glove dispensing bays for a first size (e.g., small) 704/704, a second size (e.g., medium) 706/708, and a third size (e.g., large) 710/712

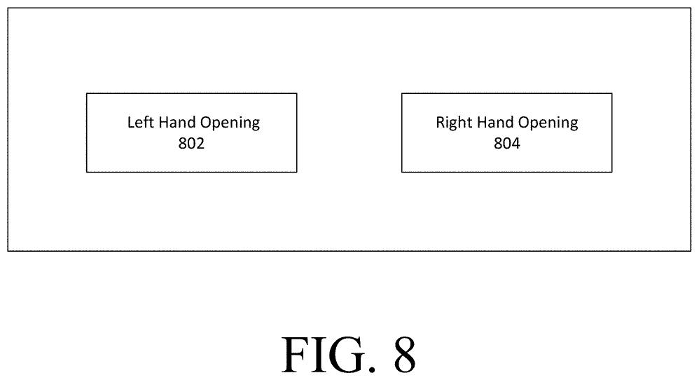

FIG. 8 is a diagram showing the front of an example glove dispenser having a single pair of glove dispensing bays 802/804.

It is, therefore, apparent that there is provided, in accordance with the various embodiments disclosed herein, methods, systems and computer readable media to dispense disposable gloves.

While the disclosed subject matter has been described in conjunction with a number of embodiments, it is evident that many alternatives, modifications and variations would be, or are, apparent to those of ordinary skill in the applicable arts. Accordingly, Applicants intend to embrace all such alternatives, modifications, equivalents and variations that are within the spirit and scope of the disclosed subject matter.

What is claimed is:

1. A glove dispenser comprising:
a housing containing:
a disposable glove supply configured to store one or more disposable gloves;
a glove dispensing bay configured to retrieve one or more disposable gloves from the disposable glove supply;
a glove opener and inflator configured to at least partially open and partially inflate the one or more disposable gloves received in the glove dispensing bay; and
a control unit configured to control the glove dispenser.

2. The glove dispenser of claim 1, wherein the control unit is configured to perform operations including:
detecting when a user inserts one or both hands into the glove dispensing bay;
prior to the user inserting one or both hands into the glove dispensing bay, causing a glove opener and inflator to retrieve one or more gloves from the disposable glove supply; and
as the one or more hands of the user enters the glove dispensing bay, causing the glove opener and inflator to mechanically open the one or more gloves at least partially and causing the inflator to blow air into the glove to partially inflate the one or more gloves such that the user is aided in inserting the one or more hands into the one or more gloves.

3. The glove dispenser of claim 2, wherein the air used to at least partially inflate the one or more gloves is supplied by an air source comprising one of an air pump or a compressed air source.

4. The glove dispenser of claim 1, further comprising an ultraviolet (UV) or other proximity sensor to detect proximity of the one or more hands of a user.

5. The glove dispenser of claim 1, wherein the glove dispenser is configured to dispense a single size of disposable glove.

6. The glove dispenser of claim 1, wherein the glove dispenser is configured to dispense multiple sizes of disposable gloves.

7. A method to operate and control a disposable glove dispenser, the method comprising:
detecting, at the disposable glove dispenser, a user's hand approaching or entering a glove dispensing bay of the disposable glove dispenser;
loading one or more disposable gloves into the glove dispensing bay by mechanically retrieving the one or more gloves from a disposable glove supply;
partially opening the one or more disposable gloves are partially opened;
activating an inflation air source to partially inflate the one or more gloves;
detecting a withdrawal of the one or more hands of the user from the glove dispensing bay; and
deactivating the inflation air source.

8. The method of claim 7, further comprising resetting the disposable glove dispenser for a next dispensing operation.

9. The method of claim 7, further comprising receiving, at the disposable glove dispensing system a glove size selection.

10. The method of claim 9, wherein the glove size selection is received from one of a control panel, a voice command, or by inserting the one or more hands into a slot associated with a particular size of disposable glove.

11. The method of claim 7, wherein the disposable glove dispenser includes a UV or other proximity sensor to detect the proximity of the one or more hands of the user.

12. The method of claim 7, wherein the disposable glove supply is within the disposable glove dispenser.

13. The method of claim 7, wherein the disposable glove supply is external to the disposable glove dispenser.

14. The method of claim 7, wherein the partial opening is performed mechanically.
